(19) United States
(12) Patent Application Publication
(10) Pub. No.: US 2026/0179764 A1
(43) Pub. Date: Jun. 25, 2026

(54) SYSTEM AND METHOD FOR ALLOCATING MEDICAL STAFF TO A NUMBER OF MEDICAL DEVICES

(71) Applicant: Siemens Healthineers AG, Forchheim (DE)

(72) Inventors: Pratyush ROUT, Bangalore (IN); Manivannan SUNDARAPANDIAN, Bangalore (IN); Janina BEILNER, Muenchen (DE)

(73) Assignee: Siemens Healthineers AG, Forchheim (DE)

(21) Appl. No.: 19/428,672

(22) Filed: Dec. 22, 2025

(30) Foreign Application Priority Data
Dec. 23, 2024 (EP) 24222782.5

(51) Int. Cl. G16H 40/20; A61B 34/00; G16H 40/63

(52) U.S. Cl. CPC G16H 40/20; G16H 40/63; A61B 34/25

(57) ABSTRACT

A system for allocating medical staff to a first number of medical devices and for controlling the first number of medical devices comprises a user interface configured to acquire input data and adding the acquired input data to a set of constraints; a scheduler unit configured to provide a second number of schedules by allocating a third number of allocated medical staff out of a fourth number of total medical staff to at least one of the first number of medical devices or a working shift based on the set of constraints; and the first number of medical devices, wherein the system is configured to control the first number of medical devices based on the provided second number of schedules.

### CROSS-REFERENCE TO RELATED APPLICATION(S)

The present application claims priority under 35 U.S.C. § 119 to European Patent Application No. 24222782.5, filed Dec. 23, 2024, the entire contents of which is incorporated herein by reference.

### FIELD

One or more example embodiments relates to a system for allocating medical staff to a number of medical devices, a method, and a computer program product.

### RELATED ART

In medical facilities, for example, in medium to large hospitals, using medical devices such as diagnostic systems or surgeon systems controlling and scheduling the medical devices as well as scheduling the medical staff using the medical devices is a challenging problem, that is, generating a usage scheme or schedule defining when, where, in which manner and by whom of the medical staff each of the medical devices of the medical facility will be used in the future. Said provides a workflow instruction for the medical facility, eventually influencing the manner how and when the medical devices will be used. In particular, in case scheduling reaches across various working shifts within the medical facility and/or across various departments of the medical facility, said scheduling is a challenging problem. Therefore, optimal controlling and scheduling of the medical staff and the medical devices is a key topic of interest in healthcare management, as it directly impacts the operational efficiency of a hospital. Independent of the grammatical term usage, individuals with male, female or other gender identities are included within the term.

### SUMMARY

Usually, the scheduling is done by a unit manager for a single planning horizon, covering for example the next four weeks. For solving said scheduling, an automated scheduling software can be used in order to ease achieving a transparent and unbiased solution. However, as the number of the medical staff members, the number of medical devices, and/or the number of working shifts scale up, the problem can become impossible or time-consuming to solve in a satisfying manner, that is, with respect to possible given constraints. With the classical computing hardware, depending on problem complexity, the automated scheduling software can take several tens of minutes or more to generate a schedule for a single planning horizon. Furthermore, within automated scheduling software, the underlying model to generate the schedule cannot be tuned directly by the unit manager in order to minimize possible constraint violations. Hence, typically, the unit manager has to employ trial and error methods to tune the model which is inefficient and time-consuming and can cause an inadequate usage of the medical devices and/or an inadequate treatment of the patients. In addition, it is beneficial to allocate the medical staff to the medical devices in a balanced manner to achieve the best efficiency of the medical staff and/or the medical devices. That is, the members of the medical staff have to be allocated across various medical devices according to their individual different knowledge levels and experience with respect to every medical device. Hereby, the members of the medical device gain experience in using every medical device in a balanced manner. This can provide a safe, adequate, and efficient usage of the medical devices and an adequate treatment of the patients. Nonetheless, nowadays automated scheduling software still miss such a balanced allocation.

Therefore, one or more example embodiments improves allocating the medical staff to the number of medical devices.

### BRIEF DESCRIPTION OF THE DRAWINGS

Embodiments, features and advantages will become apparent from the subsequent description, taken in conjunction with the accompanying drawings, in which:

FIG. 1 shows a schematic illustration of a system for allocating medical staff to a number N1 of medical devices according to one or more example embodiments;

FIG. 2 shows a schematic diagram showing relations between subgroups of the medical staff for using the system of FIG. 1 according to one or more example embodiments;

FIG. 3 shows a schematic illustration of a binary search method used by the system of FIG. 1 according to one or more example embodiments; and

FIG. 4 shows a schematic block diagram illustrating method steps for using the system of FIG. 1 according to one or more example embodiments.

In the Figures, like reference numerals designate like or functionally equivalent elements, unless otherwise indicated.

### DETAILED DESCRIPTION

Accordingly, a system for allocating medical staff to a number N1 of medical devices, with N1≥1, and for controlling the N1 medical devices is suggested. The system comprises: a user interface for acquiring input data and adding the acquired input data to a set of constraints; a scheduler unit for providing a number N2 of schedules by allocating a number N3 of allocated medical staff out of a number N4 of total medical staff to the N1 medical devices and/or to a working shift based on the set of constraints, with N2≥1, N3≥1, and N4≥1; and the N1 medical devices. Hereby, the system is configured to control the N1 medical devices based on the N2 provided schedules.

The system can be configured to allocate the medical staff to the N1 medical devices in an automatic manner. In other words, after acquiring the input data, the scheduler unit can be configured to provide the N2 schedules without or with a minimized need for user interactions. The N2 schedules may comprise instructions concerning when, by whom of the medical staff and in which manner each of the N1 medical devices is used or will be used in the future. Hereby, the N2 schedules may be based on specific values of a number of variables which are limited by the set of constraints.

The N1 medical devices may comprise any system, machine, device, or tool used for medical purposes. The N1 medical devices can work autonomously and/or can be used by members of the medical staff. For example, the N1 medical devices may comprise medical imaging devices such as CT scanners, MRT scanners, MRI scanners, invivo-diagnostic machines, X-ray devices, or the like. Hereby, for a given type of medical device, the N1 medical devices may further comprise different models of the given type.

The user interface can be realized as a computer and/or as a computer terminal. The user interface can comprise a desktop, a monitor, a touch screen, a keyboard, a mouse, and/or any other input or control device. Using the user interface, the user may communicate with the system, and both insert input data into the system as well as receive output data from the system. Hereby, the input data may comprise any data required to provide the N2 schedules, for example, information concerning the N4 total medical staff, such as the number N4, that is, how many members of the medical staff are available in total, the number of staff members being required within each working shift of the medical facility, the respective experience level of each member of the N4 total medical staff regarding each of the N1 medical devices, the planned working hours and days off of the medical staff members, etc. The input data may further comprise information regarding the N1 medical devices, for example, a list of all N1 medical devices, their availability in total, within specific working shifts, and/or for each day or week within each planning horizon covered by the N2 schedules. The input data may further comprise the required number and/or the required experience level of medical staff members that is required in order to operate a respective one of the N1 medical devices. In addition, the input data may further comprise additional constraints such as pending medical device malfunctions or the like.

The acquired input data may be added to a set of constraints. It is further feasible that the set of constraints may comprise internal data of the system, for example, data of previously provided schedules and/or information regarding the N1 medical devices. The set of constraints may represent entries of at least one database. The set of constraints may comprise all constraints required to provide the N2 schedules, hence, the set of constraints may represent all factors and/or limitations which need to be respected in order to generate the N2 schedules. Hereby, some of the constraints may have to be fulfilled completely while other constraints may have to be fulfilled as far as possible. For example, the constraints may comprise time slots when each of the N1 medical devices is available, the duration how long each of the N1 medical devices can be used in a row, the duration how long a specific usage of each of the N1 medical devices takes, the number of medical staff members being required for each of the N1 medical devices with respect to specific usages of the N1 medical devices, the weekdays and/or times a specific staff member should work, the number of days off of a specific staff member during one planning horizon, and/or others.

The scheduler unit may be a computer and/or a computer program product which is configured to provide the N2 schedules based on the set of constraints. Hereby, the scheduler unit may be configured to provide the N2 schedules in the frame of an iterative calculation process within which the scheduler unit is configured to aim on finding an optimal solution for respecting the set of constraints. The scheduler unit may comprise and/or may be configured to apply algorithms for providing the N2 schedules and/or respective machine learning tools. Each of the N2 schedules may cover one or more planning horizons. Hereby, each planning horizon may cover a future time window of one week, one month, one year, etc. Each of the N2 schedules may comprise instructions when and how each of the N1 medical devices is used within the respective planning horizon as well as by which one or more members of the N4 total medical staff.

The N4 total medical staff may represent all available staff members using the system, for example, all staff members of a medical department or of the whole medical facility. The N3 allocated medical staff may represent a subgroup of the N4 total medical staff. The N3 allocated medical staff may represent those of the N4 total medical staff who are allocated to the N1 medical devices in the frame of the provided N2 schedules. In other words, the N3 allocated medical staff may represent the subgroup of the N4 total medical staff which is selected to work due to the N2 schedules. Hereby, the N3 allocated medical staff may be allocated both regarding the N1 medical devices, meaning where each member of the N3 allocated medical staff will work according to the provided N2 schedules, as well as when to work at each of the N1 medical devices. Furthermore, the N3 allocated medical staff may be allocated across working shifts, meaning the provided N2 schedules define within which working shift each member of the N3 allocated medical staff will work as well as within which team and/or department of the medical facility. The working shifts may comprise, for example, a morning shift, an afternoon shift, and/or a night shift. It is further possible that the N3 allocated medical staff may be allocated according to additional dimensions with respect to their individual skill level, experience level, etc.

Controlling the N1 medical devices may comprise controlling the manner when, how, and by which staff member each of the N1 medical devices is used in the frame of the provided N2 schedules. Furthermore, controlling the N1 medical devices may comprise executing instructions concerning which operation or operations have to be performed by each medical device and in which manner. Hereby, the N1 medical devices may be controlled directly by the system, for example, by sending instructions and/or other data from the scheduler unit to the N1 medical devices. This way, the system interacts directly with the N1 medical devices and controls when and how they are operating. In addition, or alternatively, the system may be configured to provide instructions to the user regarding the controlling of the N1 medical devices. Said may be realized by visualizing the instructions on a monitor, a display, and/or the like and/or by printing the instructions using a printer, a plotter, and/or the like.

The system may enable the generation of the N2 schedules in brief time, for example within a time window of several seconds or minutes. This way, the N2 schedules may be updated regularly and/or when facing specific events like malfunctions and/or maintenance actions of the N1 medical devices, and/or illnesses and/or vacations of members of the N4 total medical staff. Furthermore, the system may be able to pay respect to a number of constraints when providing the N2 schedules. In addition, the system may allow the user to influence the generation of the N2 schedules in an easy and/or pinpointed manner, for example, by adapting the constraints. Eventually, the N1 medical devices may be operated in a more efficient manner and/or with a lower probability of malfunctions. For example, time slots with an idle state of one or more of the N1 medical devices caused by an unavailability of members of the medical staff and/or an inadequate usage of the N1 medical devices may be avoided.

According to an embodiment, the set of constraints comprises a number N5 of hard constraints, a number N6 of soft constraints, and/or weightings of the N6 soft constraints, with N5≥1 and N6≥1.

The N5 hard constraints are mandatory. That is, the N5 hard constraints have to be completely fulfilled in order to provide the N2 schedules. Any violation of one of the N5 hard constraints due to the N2 schedules prevents the scheduler unit from providing the N2 schedules to the user. In other words, the scheduler unit is configured to adjust the N2 schedules until each of the N5 hard constraints is fulfilled. The N5 hard constraints can comprise, for example, a given availability of the N1 medical devices, a given shift rotation of the N4 total medical staff, that is, for example, that after a staff member has been working within a specific working shift for one or more weeks, the respective staff member will work in another working shift in the subsequent week. The N5 hard constraints may further comprise other mandatory requirements, for example a certain number of staff members being required to work with a specific medical device at a given time and/or for a given operation of the specific medical device. In contrast, the N6 soft constraints are desirable but optional. In other words, an increased degree of fulfilling of the N6 soft constraints may improve the quality of the N2 schedules. Nonetheless, a possible violation of the N6 soft constraints still enables providing the N2 schedules. The N6 soft constraints can comprise, for example, the requirement to provide a specific number of days off per week for each staff member, a specific number of hours a given staff member should work within a specific week, etc. The weightings can be set by the user and/or the system and can define the importance of each of the N6 soft constraints. The higher the importance of a given one of the N6 soft constraints is, the more the scheduler unit aims on fulfilling the given soft constraint when providing the N2 schedules, for example, within the iterative calculation process. In other words, with a rising weighting, the fulfilling of the given soft constraint is more likely. Hereby, the weightings can define the importance of each of the N6 soft constraints in relation to the respective other ones of the N6 soft constraints. In addition, or alternatively, the weightings can define the importance of the N6 soft constraints as a whole in relation to the N5 hard constraints. The weightings can be updated while using the system. For example, the weighting of a given one of the N6 soft constraints can be updated according to a number of times the given soft constraint is selected for adjustment by the user. In addition, or alternatively, the weighting of the given soft constraint can be updated according to an extent of violation of a value and/or range of the given soft constraint which is defined as being acceptable by the user. Each one of the N6 soft constraints can be addressed with a specific weighting. The user is provided with an easy manner for influencing the N2 schedules by adjusting the weightings and/or the allowable ranges of the N6 soft constraints. The weightings can be stored within a local memory of the system and/or on a cloud server.

According to a further embodiment, the N6 soft constraints comprise a required allocation of the N4 total medical staff to the N1 medical devices, in particular, the weightings comprise a weighting of the required allocation in relation to the others of the N6 soft constraints.

The required allocation refers to the time window of the provided N2 schedules, in particular, to the time window of a consecutive next one of the N2 provided schedules. The required allocation represents such an allocation of the N4 total medical staff to the N1 medical devices which is desired in order to converge towards or to achieve a balanced allocation over time. Hereby, the balanced allocation can be understood such way that when achieving and/or when proceeding a balanced allocation, every one of the N4 total medical staff has the same level of experience concerning the usage of a specific one of the N1 medical devices. In other words, the allocation of the N4 total medical staff may change over time such way that every one of the N4 total medical staff gains experience in using every one of the N1 medical devices, in particular, simultaneously. In addition, or alternatively, the balanced allocation can be understood as such allocation, according to which staff members with higher levels of experience are assigned to work with staff members with lower levels of experience regarding a specific medical device in order to provide a perspective transfer of experience. Hence, when a given one of the N4 total medical staff has low experience in using a given one of the N1 medical devices, the required allocation of the provided N2 schedules comprises an increased allocation of the given staff member to the given medical device, for example, by an increased intensity and/or time duration of the usage of the given medical device by the given staff member. This way, the experience of the given staff member in using the given medical device is increased over time. Therefore, the higher N2 is, that is, the more planning horizons the provided N2 schedules cover, the more precisely the balanced allocation can be achieved. With the N6 soft constraints comprising the required allocation, the N2 schedules can be adapted according to the level of experience of each of the N4 total medical staff, providing a learning effect for each of the N4 total medical staff according to the current level of experience. Eventually, with increasing levels of experience, an increased quality and efficiency of the usage of the N1 medical devices can be achieved.

According to a further embodiment, the scheduler unit is configured to provide the N2 schedules by: generating a first schedule of the N2 schedules based on the set of constraints, in particular by setting a predefined initial distribution for a machine distribution constraint, wherein the machine distribution constraint is one of the N6 soft constraints; displaying the generated first schedule to a user of the system; optionally improving the generated first schedule; and calculating an allocation error of each of the N6 soft constraints based on a deviation between a scheduled allocation of the first schedule and the required allocation. In particular, the scheduler unit is configured to provide the N2 schedules by adjusting the weightings for at least one further schedule of the N2 schedules and repeating steps above for the at least one further schedule.

Hereby, the first schedule can cover one planning horizon, for example one week or one month. The machine distribution constraint can be a quantitative and/or qualitative information concerning how each member of the N4 total medical staff was allocated to the N1 medical devices in the past. Hence, the machine distribution constraint can represent a skill level for each one of the N4 total medical staff regarding each one of the N1 medical devices. The machine distribution constraint can be a vector, an array, or a matrix. For generating the first schedule, the machine distribution constraint can be set to the predefined initial distribution. The predefined initial distribution can be, for example, to set a uniform distribution, thus, setting uniform values for all entries of the machine distribution constraint, representing a uniform skill level of each of the N4 total medical staff regarding each one of the N1 medical devices. By improving the generated first schedule, the user has the possibility to modify the generated first schedule if necessary. To improve the generated first schedule can mean to adjust the generated first schedule such way that the generated first schedule meets the set of constraints with fever violations and/or with lower degrees of violation. For example, the user can adjust the set of constraints, in particular, the N6 soft constraints, for example, by adjusting the allowable ranges of the N6 soft constraints and/or by adjusting the weightings of the N6 soft constraints, followed by a regeneration of the first schedule. Possible violations of the N6 soft constraints can cause deviations between the scheduled allocation of the first schedule and the required allocation. In other words, with the calculated first schedule, the balance allocation is not met precisely. Hereby, the scheduled allocation represents the allocation of the N3 allocated medical staff to the N1 medical devices as calculated by the scheduler unit for a given planning horizon and/or within the generated first schedule. The deviation of each of the N6 soft constraints results in the calculated allocation error which describes the degree of the respective deviation.

In the following, one example of a method for calculating the allocation error and adjusting the weightings is described. Let the N1 medical devices to be used within a given planning horizon and/or within the N2 schedules be [M1, M2, M3, . . . ]. Furthermore, the number of staff members required in order to operate the N1 medical devices can be [t1, t2, t3, . . . ]. This way, the number, Rᵢₖ, of working slots to be assigned for each staff member, i, during which the staff member i is working with a medical device, k, can be given as Rᵢₖ=[t1/N3, t2/N3, t3/N, . . . ] with N3 as the number of allocated medical staff for the given planning horizon and/or the N2 schedules. This way, Rᵢₖ describes a required allocation value for the i^{th} staff member in the k^{th} medical device and represents the ideal case. In other words, with Rᵢₖ, a balanced allocation can be achieved. Initially, before the calculation of the first schedule, a weight, Wᵢₖ, of each of the N6 soft constraints can be set to be 1. Hereby, uniform weights for a medical device distribution constraint, MDDC, which can be one of the N6 soft constraints, are set. The weightings of the N6 soft constraints can comprise the weight Wᵢₖ. Furthermore, initially, the allocation error, eᵢₖ, regarding an allocation of the staff member i to the medical device k, can be set to be 0. Then, by using an allocation matrix,

$${X}_{ij} = \{ \begin{matrix}1 & if staff member i allocated to a shift j \\ 0 & otherwise\end{matrix} \} ,$$

the medical device distribution constraint MDDC can be calculated by MDDC=Σ_{j∈J}_{ₖ}Xᵢⱼ=Rᵢₖ+eᵢₖ, ∀i ∈N3, K∈N1 with N1 as the number of medical devices and Jₖ as a set of shifts belonging to the medical device k. Let the scheduled allocation as generated by the scheduler unit be Sᵢₖ describing an allocation of the staff member i to the medical device k. Then, the allocation error eᵢₖ can be calculated as eᵢₖ=Sᵢₖ−Rᵢₖ. For the calculation of a further planning horizon and/or a further schedule of the N2 schedules, the weight Wᵢₖ can be adjusted and set to be Wᵢₖ=1+α·abs(eᵢₖ) with abs (eᵢₖ) giving the absolute value of the allocation error eᵢₖ and α representing a scaling parameter which can be tuned, for example during usage of the system by the scheduler unit and/or by the user. Nonetheless, said example for calculating the allocation error and adjusting the weightings given above is of exemplary manner. Other methods are feasible as well.

Eventually, with the allocation error, a numerical and quantitative value is provided defining the quality of the provided N2 schedules, in particular, of the first schedule with respect to achieving the balanced allocation. In other words, with the allocation error, the scheduler unit and/or the user can evaluate how well the generated first schedule meets the balanced allocation. Possibly in response to the calculated allocation error, the scheduler unit and/or the user can adjust the weightings of the N6 soft constraints, for example, for the calculation of the at least one further schedule of the N2 schedules, followed by a subsequent generation of the at least one further schedule of the N2 schedules. Hereby, the at least one further schedule can cover at least one subsequent planning horizon, for example the respective next week(s) or next month(s).

According to a further embodiment, the scheduler unit is configured to improve the first schedule by: selecting at least one of the N6 soft constraints for improvement by the user; adjusting an allowable violation of the selected at least one of the N6 soft constraints by the user or performing an automatic adjustment of the N6 soft constraints to find a minimum violation of the selected at least one of N6 soft constraints; and updating the first schedule. In particular, the scheduler unit is configured to improve the first schedule by repeating the above-mentioned steps for another one of the N6 soft constraints.

The selected at least one of the N6 soft constraints represents the one or more which shall be improved. For example, the selected at least one soft constraint can be the least weighted one of the N6 soft constraints. Hereby, the improvement can be understood in the sense of reducing possible violations of the selected at least one soft constraint or, which is preferred, of the violations of all of the N6 soft constraints as a whole, that is, an improvement of the overall N2 schedules. The scheduler unit can be configured to keep constant those of the N6 soft constraints and/or respective values addressed with a higher weighting than the selected at least one soft constraint. This way, by adjusting the allowable violation of the selected at least one soft constraint higher rank soft constraints are not altered. The allowable violation can refer to a quantitative and/or qualitative range concerning a given soft constraint in which respective values of the generated N2 schedules have to be located in. A placement of value concerning a given soft constraint outside of said range by the scheduler unit when generating the N2 schedules is considered as a violation of the given soft constraint. For example, the selected at least one soft constraint can represent the working hours per day of a given staff member. The respective allowable violation of that soft constraint may be a range of 6 to 9 working hours per day. This way, when generating the first schedule, the scheduler unit will aim achieve between 6 to 9 working hours for the given staff member for every day, on which the given staff member is working at the medical facility. At the same time, the scheduler unit aims to pay respect to all other hard constraint and soft constraints, possibly causing significant violations of other soft constraints which can lower the overall quality of the generated first schedule. In case the generated first schedule is of poor quality and/or shows many violations of the set of constraints, the user can select the above-mentioned soft constraint representing the working hours per day of the given staff member and, for example, enlarge the range by allowing between 5 to 10 hours per day. A consecutive regeneration of the first schedule can show the effect of said adjustment. In case the generation of the first schedule is not possible with said adjustment, for example, since one or more of the N5 hard constraints cannot be fulfilled anymore, the system can notify the user about that. The adjustment of the selected one of the N6 soft constraints and the consecutive regeneration of the first schedule can be understood as a single optimization step. Alternatively, the system can be configured to perform the automatic adjustment of the N6 soft constraints. There, the scheduler unit can be configured to automatically find a minimum violation of the selected at least one soft constraint by performing one or more optimization steps. The scheduler unit can be configured to perform a binary search method in order to reduce the violation ranges of the N6 soft constraints. Nonetheless, other methods for identifying the minimum violation of the selected at least one soft constraint are feasible, as well. If desired by the user, the above-mentioned optimization steps can be repeated for another one of the N6 soft constraints in order to further improve the first schedule. The above-mentioned optimization steps are described for the generation of the first schedule. Nonetheless, said optimization steps can be applied in order to improve any further one of the N2 schedules, as well. In general, throughout the whole application, everything mentioned regarding the first schedule can be valid for every at least one further schedule, as well. Furthermore, at least one of the above-mentioned steps can be automatically performed by the scheduler unit, as well.

According to a further embodiment, the scheduler unit is configured to respond to the adjustment of the allowable violation by the user by performing a single optimization step while keeping the violation of those of the N6 soft constraints with a higher weighting than the selected at least one of the N6 soft constraints.

The N6 soft constraints with a higher weighting than the selected at least one of the N6 soft constraints can represent, for example, such soft constraints which are considered to be more important than other soft constraints. This way, when performing the single optimization step, the scheduler unit can be configured to adjust the values of such soft constraints with a lower weighting first while the ones with a higher weighting remain constant or are adjusted afterwards. This approach provides a minimal-invasive optimization of the first schedule.

According to a further embodiment, the scheduler unit is configured to perform the automatic adjustment of the N6 soft constraints during multiple optimization steps using a binary search method, in particular, by iteratively reducing violation ranges.

When performing the binary search method, in a first optimization step, the scheduler unit is configured to split an initial violation range of a given soft constraint into two halves. The scheduler unit is configured to identify in which of the halves an optimal solution is located. The identified half becomes a second violation range for a second optimization step. Then, the scheduler unit is configured to split the second violation range again into two halves, and so on. Hereby, the number of optimization steps is arbitrary. The binary search method provides a fast optimization of the first schedule by finding the minimum violations rapidly.

According to a further embodiment, the scheduler unit is configured to provide the N2 schedules such way that each member of the N4 total medical staff is assigned to every one of the N1 medical devices in a balanced manner.

Hereby, a balanced allocation can be achieved. Note that the precision of the balanced allocation can be improved with the number N2. That is, the more schedules are generated, the more precisely the balanced allocation can be achieved. This way, a gain in experience in using every one of the N1 medical devices by every member of the N4 total medical staff can be achieved.

According to a further embodiment, the scheduler unit comprises a schedule database and/or a constraints database.

Previously generated schedules can be stored within the schedule database for reference, comparison, etc. Furthermore, after their generation, the generated N2 schedules can be stored within the schedule database. In a similar way, previous constraints and their weightings, which have been used for generating the previously generated schedules, can be stored within the constraints database for reference, comparison, etc. After their acquisition, the N6 soft constraints and the weightings can be stored within the constraints database, as well. The previously generated schedules and/or the respective previous constraints and weightings can be used for generating the N2 schedules. For example, the weight of a given soft constraint can be calculated according to the number of times the given soft constraint has been selected in the past for updating the violation range concerning the given soft constraint. In addition, the weight of the given soft constraint can be calculated according to the size of the violation range of the given soft constraint. That is, for example, the smaller or narrower the violation range, the higher the weight of the given soft constraint. Nonetheless, the user can explicitly set the weight of a given soft constraint, as well. With said databases, the system is able to learn from previous usages of the system. The system can use previous schedules, constraints, and/or weightings for the generation of the N2 schedules, minimizing the required input data and/or other user interactions and, eventually, utilize gained knowledge in using the N1 medical devices for improving the future usage of the N1 medical devices and saving time and costs.

According to a further embodiment, the system further comprises a local memory and/or a cloud server, wherein the scheduler unit is configured to acquire the set of constraints and/or the weightings from the user interface, the local memory, and/or the cloud server. In particular, the scheduler unit is configured to read and/or to store the schedule database and/or the constraints database from/to the local memory and/or the cloud server.

The local memory can be a hard drive or any other memory device being integrated within the system. The cloud server can be a server or any other memory device being addressed via an intranet connection, internet connection, and/or other connection means. This way, the system can use previous schedules and/or constraints for the generation of the N2 schedules, minimizing the required input data and/or other user interactions and, eventually, utilize gained knowledge in using the N1 medical devices for improving the future usage of them and saving time and costs. This way, the schedule database and/or the constraints database are usable by other systems, as well.

According to a further embodiment, the scheduler unit comprises a solver unit for providing the N2 schedules, wherein the solver unit is configured to apply an optimization algorithm. In particular, the solver unit is configured to apply a constrained quadratic model solver from a D-Wave library. Furthermore, in particular, the solver unit is configured to compute the optimal set of decision variables using a quantum annealing technique.

In addition, or alternatively, other optimization algorithms can be applied by the solver unit in order to provide the N2 schedules, as well. Hereby, the solver unit can be a computer program product and/or a computer.

According to a further embodiment, the N2 schedules span over N2 planning horizons.

For example, the N2 schedules can span over one, two, three, four, five, or more planning horizons.

According to a further embodiment, the solver unit is configured to minimize a gap between a number of N7 required medical staff and the N3 allocated medical staff.

The N7 required medical staff can represent the minimal number of staff members required to operate the N1 medical devices. Hence, the solver unit can be configured to assign a number of staff members to each of the N1 medical devices as close as possible to the respective required number of medical staff member being required to operate the respective medical device. Hereby, the required number of staff members can be individual for each medical device. Furthermore, the required number of staff members of a given medical device can vary according to the type of the given medical device and/or the planned type of usage of the given medical device.

According to a further embodiment, the scheduler unit is configured for acquiring input data of the N1 medical devices.

The scheduler unit can be configured to receive data and/or instructions from N1 medical device, for example, addressing the previous and/or current usage of the N1 medical devices by the N3 allocated staff. If, for example, the N1 medical devices detect an improper and/or inefficient usage by the N3 allocated staff, the scheduler unit can be informed and/or instructed to vary the allocation of the N4 total medical staff accordingly, for example, in the next schedule and/or planning horizon. The user can be advised and/or informed concerning that via the user interface. This way, the efficiency of the N1 medical devices can be further improved.

According to a further aspect, a method for using a system with a number N1 of medical devices is suggested. Hereby, the above-mentioned system is particularly embodied according to the above described aspect or according to any embodiment of the above described aspect. The method comprises: acquiring input data by a user interface and adding the acquired input data to a set of constraints; providing a number N2 of schedules based on the acquired input data by a scheduler unit, with N2≥1; outputting the provided N2 schedules to a user by the user interface; and controlling the N1 medical devices based on the N2 provided schedules.

According to a further aspect, a computer program product is suggested comprising a program code for executing the above-mentioned method for when run on at least one computer.

The computer program product, such as a computer program means, may be embodied as a memory card, USB stick, CD-ROM, DVD or as a file which may be downloaded from a server in a network. For example, such a file may be provided by transferring the file comprising the computer program product from a wireless communication network.

The embodiments and features described with reference to the system and/or the computer program product apply mutatis mutandis to the method according to one or more example embodiments.

Further possible implementations or alternative solutions of the invention also encompass combinations—that are not explicitly mentioned herein—of features described above or below with regard to the embodiments. The person skilled in the art may also add individual or isolated aspects and features to the embodiments.

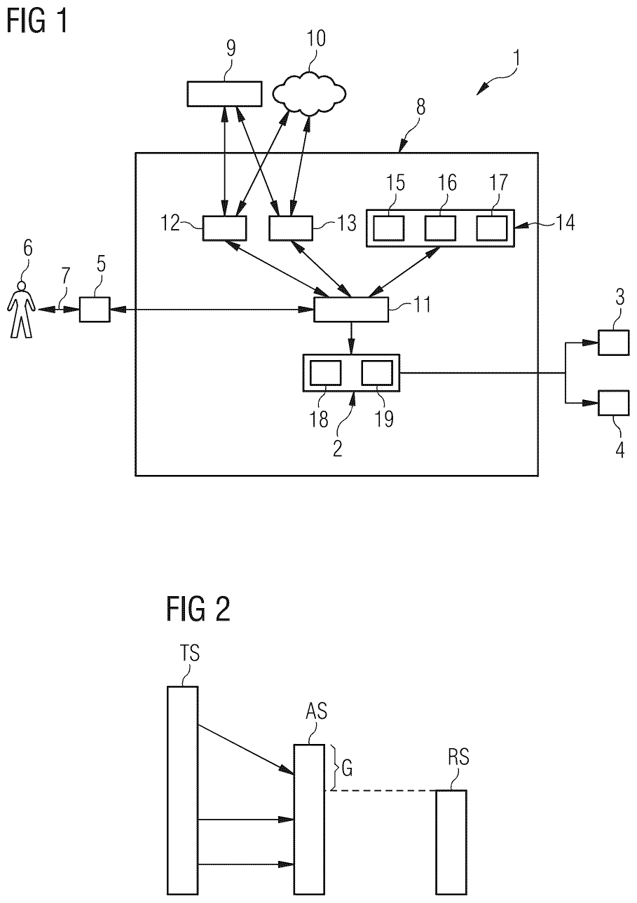

FIG. 1 shows a schematic illustration of a system 1 for allocating medical staff to a number N1 of medical devices 3, 4, with N1≥1. The system 1 is configured to generate a number N2 of schedules 2, with N2≥1, by allocating the medical staff to the N1 medical devices 3, 4. That is, to define when and how each of the N1 medical devices 3, 4 is used by which one or more members of the medical staff. This way, the manner of usage of the N1 medical devices 3, 4 is defined.

The system 1 can be located in a medical facility (not shown) in order to allocate the medical staff related to the medical facility to the N1 medical devices 3, 4 related to the medical facility. Hereby, both the number of medical staff members as well as the number N1 of medical devices 3, 4 are arbitrary. In the following, for visualization reasons, only two medical devices 3, 4 are shown.

The system 1 comprises a user interface 5 to enable a user interaction between a user 6 using the system 1 and the system 1. The user interface 5 is configured to receive input data 7 from the user 6 and/or to output data to the user 6.

The system 1 further comprises a scheduler unit 8. The scheduler unit 8 can be implemented as a computer program product or represent a computer (not shown). The scheduler unit 8 is in communication with the user interface 5. Furthermore, the scheduler unit 8 is in communication with a local memory 9 and/or a cloud server 10. The local memory 9 can be a hard drive of the computer (not shown) on which computer the system 1 is implemented. The scheduler unit 8 is configured to transfer data via the Internet and/or to store data on the cloud server 10.

The scheduler unit 8 comprises a solver unit 11 in order to process the input data 7, the data of the local memory 9, and/or the cloud server 10. The solver unit 11 can be configured to apply an optimization algorithm, in particular, a constrained quadratic model solver from a D-Wave library. For example, the solver unit 11 can be configured to allocate the medical staff members to the N1 medical devices 3, 4 by computing an optimal set of decision variables using a quantum annealing technique.

The scheduler unit 8 comprises a constraints database 12 on which previous constraints (not shown) and/or previous weightings (not shown) from previous usages of the system 1 can be stored. Constraints being part of the input data 7 can be stored in the constraints database 12 as well. In addition, the scheduler unit 8 comprises a schedule database 13 for storing schedules previously generated by the scheduler unit 8.

The scheduler unit 8 further comprises a set of constraints 14. The set of constraints 14 comprises all constraints relevant for generating the N2 schedules 2. That is, all factors defining degree of freedoms inside which the solver unit 11 can produce solutions for the N2 schedules 2. The scheduler unit 8 is configured to save all constraints received via the input data 7 as well as relevant constraints from the constraints database 12 within the set of constraints 14. Hereby, the constraints are subdivided according to their importance. Eventually, the set of constraints 14 comprises a number N5 of hard constraints 15, with N5≥1, which are mandatory to fulfill when generating the N2 schedules 2, as well as a number N6 of soft constraints 16, with N6≥1, which are optional but desired to fulfill. In addition, the set of constraints 14 comprises weightings 17 which define the importance of each of the N6 soft constraints 16 in relation to the respective other N6 soft constraints 16 as well as the relevance of the N6 soft constraints 16 as a whole in relation to the N5 hard constraints 15. The scheduler unit 8 is configured to generate the N2 schedules 2 based on the set of constraints 14. The N2 schedules 2 comprise a first schedule 18 which is generated first, as well as optionally, at least one further schedule 19 which is generated after the first schedule 18. Hence, the existence of the at least one further schedule 19 is of exemplary manner. The system 1 can be used to generate solely the first schedule 18, as well. The number of further schedules 19 is arbitrary. Each of the first schedule 18 and the at least one further schedule 19 can cover one planning horizon, for example one week or one month.

FIG. 2 shows a schematic diagram showing relations between subgroups of the medical staff for using the system 1.

The medical staff of the medical facility can be subdivided into a number N4 of total medical staff TS, N4≥1, comprising all medical staff members related to the medical facility. When generating the N2 schedules 2, the solver unit 11 is configured to select a number N3 of allocated medical staff AS, with N3≥1. The N3 allocated medical staff AS represent those members of the N4 total medical staff TS which are scheduled to work with the N1 medical devices 3, 4 in the frame of the generated N2 schedules 2. In other words, possibly, not every member of the N4 total medical staff TS may be allocated to work with one of the N1 medical devices 3, 4 within the planning horizons covered by the N2 schedules 2. Therefore, the number N3 is similar or smaller than the number N4. In FIG. 2, said selection process performed by the solver unit 11 is illustrated by three arrows.

The medical staff can further comprise a subgroup with a number N7 of required medical staff RS, N7≥1, representing an ideal number of medical staff members which are required in order to operate the N1 medical devices 3, 4. When selecting the N3 allocated medical staff AS, the solver unit 11 is configured to allocate at least the N7 required medical staff RS. That is, to provide the N1 medical devices 3, 4 with at least as many medical staff members as required in order to operate the N1 medical devices 3, 4. On the other hand, in case the solver unit 11 allocates more medical staff members to the N1 medical devices 3, 4 as required, a gap G is formed, representing an overrepresentation of the N3 allocated medical staff AS. For economic and efficiency reasons, the solver unit 11 can be configured to minimize the gap G when generating the N2 schedules 2.

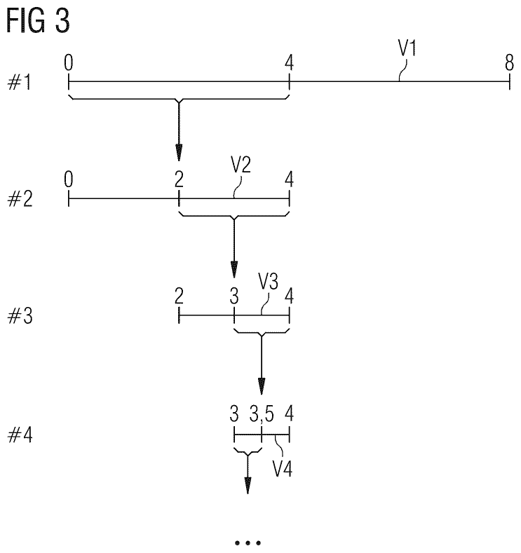

FIG. 3 shows a schematic illustration of a binary search method used by the system 1.

When generating the N2 schedules 2, the solver unit 11 is configured to allocate the medical staff members to the N1 medical devices 3, 4 while fulfilling all of the N5 hard constraints 15 as well as fulfilling the N6 soft constraints 16 as much as possible. Possible violations of the N6 soft constraints 16 are allowed but shall be avoided. In order to minimize the violations of the N6 soft constraints 16, the solver unit 11 can be configured to automatically identify an optimum regarding a given soft constraint 16 using the binary search method. Hereby, in a first step #1, an initial violation range V1 of the given soft constraint 16 is split into two halves. The solver unit 11 is configured to identify inside which of the two halves of the initial violation range V1 the optimal solution is located, representing a second violation range V2 for a second step #2. Then, the second violation range V2 is split again into two halves, and so on. Hereby, the number of steps #1, #2, #3, etc. is arbitrary.

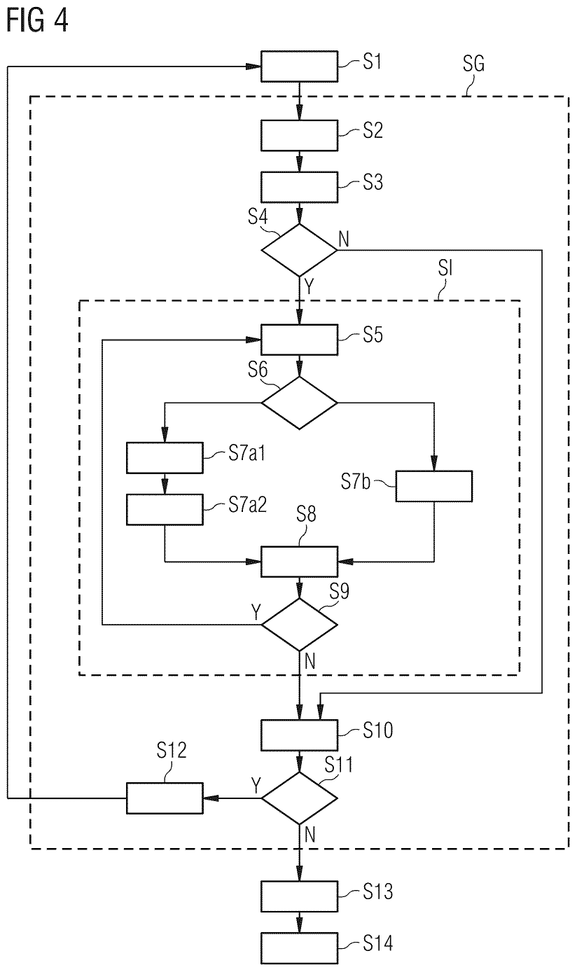

FIG. 4 shows a schematic block diagram illustrating method steps for using the system 1.

In a first step S1, the input data 7 is acquired by the user interface 5. From the user interface 5, the input data 7 can be read by the solver unit 11. Furthermore, the solver unit 11 can read previous constraints and/or previous weightings from the constraints database 12 as well as previous schedules from the schedule database 13. With said data, the solver unit 11 forms the set of constraints 14 with the N5 hard constraints 15, the N6 soft constraints 16, and the weightings 17. In a further step SG, the solver unit 11 generates the N2 schedules 2. Hereby, the step SG comprises a number of sub steps which are explained in detail in the following.

In step S2, the first schedule 18 is generated. Hereby, the first schedule 18 can cover one planning horizon, for example one week or one month. The solver unit 11 aims to fulfill all N5 hard constraints 15 as well as aims to fulfill the N6 soft constraints 16 as precisely as possible. Hereby, the solver unit 11 respects the individual weightings 17 of the N6 soft constraints 16. For generating the first schedule 18, the solver unit 11 applies an optimization algorithm, in particular, a constrained quadratic model solver from a D-Wave library (not shown). In particular, the solver unit 11 computes the optimal set of decision variables using a quantum annealing technique. The generated first schedule 18 is displayed to the user 6 together the possible violations of the N6 soft constraints 16 which occur according to the generated first schedule 18. Hereby, the user 6 is assisted in deciding whether the generated first schedule 18 is appropriate or requires further improvement. In a further step S4, the user 6 can instruct the system 1 via the user interface 5 whether a respective improvement of the generated first schedule 18 shall be performed or not. Hereby, improving can be understood as modifying the generated first schedule 18 such way that the generated first schedule 18 meets the set of constraints 14 with fever violations and/or with a lower degree of violation of the set of constraints 14. In case no further improvement is required, the method continues in step S10, which will be explained in detail below. Otherwise, the first schedule 18 is improved in step SI. Hereby, the step SI comprises a number of sub steps which will be explained in detail in the following.

In step S5, the user interface 5 enables a user selection of one of the N6 soft constraints 16 which shall be improved in order to adjust the first schedule 18. In a further step S6, the user can further decide whether the selected soft constraint 16 shall be improved by using a single run improvement (see steps S7a1 and S7a2 which will be explained in detail in the following) or by using an automatic multi-run improvement (see step S7b which will be explained in detail below).

In case the user 6 decides for an improvement by using the single run improvement, in step S7al, the user interface 5 allows an adjustment of the allowable violation range (V1, V2, V3, V4) of the selected soft constraint 16. Here, the user 6 can adjust which values of the first schedule 18 concerning the selected soft constraint 16 are defined as allowable. Hereby, for example, text inputs and/or inputs via a graphical user interface including buttons (not shown) are feasible. In step S7a2, the solver unit 11 performs a single improvement step, that is, a recalculation of the other values concerning the N6 soft constraints 16 and/or the N5 hard constraints 15 by the solver unit 11. Alternatively, in case the user 6 decides for an improvement by using the automatic multi-run improvement, in step S7b, the solver unit 11 performs multiple optimization steps for improving the violation of the selected soft constraint 16 by adjusting all other soft constraints 16 in order to identify an optimal solution for the first schedule 18. Hereby, the solver unit 11 applies the binary search method as illustrated schematically with reference to FIG. 3.

After performing the steps S7al and S7a2, or S7b, the method continues in a further step S8, where in the first schedule 18 is updated, respecting possible adjustments due to the performed optimization explained above. In a further step S9, the user 6 can influence via the user interface 5 whether a further soft constraint 16 shall be optimized or not. In case another soft constraint 16 shall be optimized, steps S5 to S8 are repeated for the further soft constraint 16. Otherwise, the method continues with step S10.

In step S10, an allocation error is calculated for the first schedule 18. Possible violations of the N6 soft constraints 16 can cause deviations between a scheduled allocation of the first schedule 18 and a required allocation. Hereby, the scheduled allocation represents the allocation of the N3 allocated medical staff AS to the N1 medical devices 3, 4 as calculated by the solver unit 11 for the generated first schedule 18. On the other hand, the required allocation represents such an allocation which allows for a balanced allocation to be achieved. The balanced allocation represents the ideal condition where every one of the N4 total medical staff TS has the same level of experience concerning the usage of a specific one of the N1 medical devices 3, 4. In other words, the allocation of the N4 total medical staff TS changes over time such way that every one of the N4 total medical staff TS gains experience in using every one of the N1 medical devices 3, 4. In addition, or alternatively, the balanced allocation can be understood as such allocation, where staff members with higher levels of experience are assigned to work with staff members with lower levels of experience in order to provide a transfer of experience. Possibly, with the calculated first schedule 18, the balance allocation is not met precisely. Then, the allocation error describes a degree of a possible deviation of the selected values for each of the N6 soft constraints 16 concerning their respective violation ranges V1, V2, V3, V4. The calculated allocation error can be shown to the user 6 via the user interface 5.

In a further step S11, the user 6 can decide by using the user interface 5 whether at least one further schedule 19 shall be generated. If so, the weightings 17 can be adjusted by the solver unit 11 according to the calculated allocation error. Hereby, specific soft constraints 16 enabling a convergence towards the balanced allocation can be matched with a higher weighting 17. Then, the steps S1 to S11 are repeated in order to generate the at least one further schedule 19. The generation of the at least one further schedule 19 follows the instructions for the first schedule 18. Hence, all explanations concerning the first schedule 18 are relevant for the at least one further schedule 19, as well. The number of further schedules 19 is arbitrary. If no further schedule 19 shall be generated, the procedure follows in step S13 which will be explained in the following. Eventually, the generated first schedule 18 and each further schedule 19 form the N2 schedules 2.

In a further step S13, the generated N2 schedules 2 are output to the user 6 using the user interface 5. For example, the user interface 5 can display the N2 schedules 2 to the user 6 and/or print the N2 schedules 2. In addition, the N2 schedules 2 are stored within the schedule database 13 and the set of constraints 14 is saved in the constraints database 12.

Finally, in step S14, the N1 medical devices 3, 4 are controlled based on the generated N2 schedules 2. That is the manner how, by whom of the N4 total medical staff TS, and in which way the N1 medical devices 3, 4 are used in the frame of the one or more planning horizons covered by the N2 schedules 2 as instructed by the N2 schedules 2. For example, the N2 schedules 2 can comprise information which operation of a given one of the N1 medical devices 3, 4 is planned for which time and/or in which order. Hence, the usage of the N1 medical devices 3, 4 is controlled by the N2 schedules 2.

Although the present invention has been described in accordance with preferred embodiments, it is obvious for the person skilled in the art that modifications are possible in all embodiments.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

### LIST OF REFERENCE

1 system
2 schedules
3 medical device
4 medical device
5 user interface
6 user
7 input data
8 scheduler unit
9 local memory
10 cloud server
11 solver unit
12 constraints database
13 schedule database
14 set of constraints
15 hard constraint
16 soft constraint
17 weightings
18 first schedule
19 further schedule
AS allocated medical staff
G gap
i staff member
j shift
J set of shifts
k medical device
M1 medical device
M2 medical device
M3 medical device
M4 medical device
M5 medical device
MDDC medical device distribution constraint
N1 number of medical devices
N2 number of schedules
N3 number of allocated medical staff
N4 number of total medical staff
N5 hard constraints
N6 soft constraints
Rᵢₖ allocation value
RS required medical staff
S1 step
S2 step
S3 step
S4 step
S5 step
S6 step
S7a1 step
S7a2 step
S7b step
S8 step
S9 step
S10 step
S11 step
S12 step
S13 step
S14 step
SG step
SI step
t1 number of staff
t2 number of staff
t3 number of staff
t4 number of staff
t5 number of staff
TS total medical staff
V1 violation range
V2 violation range
V3 violation range
V4 violation range
Wᵢₖ weight
X allocation matrix

1. A system for allocating medical staff to a first number of medical devices and for controlling the first number of medical devices, the system comprising:
a user interface configured to acquire input data and adding the acquired input data to a set of constraints;
a scheduler unit configured to provide a second number of schedules by allocating a third number of allocated medical staff out of a fourth number of total medical staff to at least one of the first number of medical devices or a working shift based on the set of constraints; and
the first number of medical devices,
wherein the system is configured to control the first number of medical devices based on the provided second number of schedules.

2. The system according to claim 1, wherein the set of constraints comprises at least one of a fifth number of hard constraints, a sixth number of soft constraints, or weightings of the sixth number of soft constraints.

3. The system according to claim 2, wherein the sixth number of soft constraints comprise a required allocation of the fourth number of total medical staff to the first number of medical devices.

4. The system according to claim 3, wherein the scheduler unit is configured to provide the second number of schedules by:
generating a first schedule of the second number of schedules based on the set of constraints;
displaying the generated first schedule to a user of the system; and
calculating an allocation error of each of the sixth number soft constraints based on a deviation between a scheduled allocation of the first schedule and the required allocation.

5. The system according to claim 4, wherein the scheduler unit is configured to improve the first schedule by:
selecting at least one of the sixth number of soft constraints for improvement by the user;
adjusting an allowable violation of the selected at least one of the sixth number of soft constraints by the user or performing an automatic adjustment of the sixth number of soft constraints to find a minimum violation of the selected at least one of N6 soft constraints; and
updating the first schedule.

6. The system according to claim 5, wherein the scheduler unit is configured to respond to the adjustment of the allowable violation by the user by performing a single optimization step while keeping the violation of those of the sixth number of soft constraints with a higher weighting than the selected at least one of the sixth number of soft constraints.

7. The system according to claim 5, wherein the scheduler unit is configured to perform the automatic adjustment of the sixth number of soft constraints during multiple optimization steps using a binary search method by iteratively reducing violation ranges.

8. The system according to claim 1, wherein the scheduler unit is configured to provide the second number of schedules such that each member of the fourth number of total medical staff is assigned to every one of the first number of medical devices in a balanced manner.

9. The system according to claim 2, wherein the scheduler unit comprises at least one of a schedule database or a constraints database.

10. The system according to claim 9, further comprising:
at least one of a local memory or a cloud server, wherein the scheduler unit is configured to acquire at least one of (i) at least one of the set of constraints or the weightings from the user interface, (ii) the local memory, or (iii) the cloud server.

11. The system according to claim 1, wherein the scheduler unit comprises:
a solver unit configured to provide the second number of schedules, wherein the solver unit is configured to apply a constrained quadratic model solver from a D-Wave library and compute an optimal set of decision variables using a quantum annealing technique.

12. The system according to claim 1, wherein the second number of schedules span over a number of planning horizons, the number of planning horizons being the second number.

13. The system according to claim 11, wherein the solver unit is configured to minimize a gap between a seventh number of required medical staff and the third number of allocated medical staff.

14. The system according to claim 1, wherein the scheduler unit is configured to acquire input data of the first number of medical devices.

15. A method for using the system of claim 1, the method comprising:
acquiring the input data by the user interface and adding the acquired input data to the set of constraints;
providing the second number of schedules based on the acquired input data by the scheduler unit;
outputting the provided second number of schedules to a user by the user interface; and
controlling the first number of medical devices based on the provided second number of schedules.

16. The system according to claim 3, wherein the weightings comprise a weighting of the required allocation in relation to others of the sixth number of soft constraints.

17. The system according to claim 4, wherein the scheduler unit is configured to provide the second number of schedules by:
generating the first schedule of the second number of schedules based on the set of constraints by setting a predefined initial distribution for a machine distribution constraint, wherein the machine distribution constraint is one of the sixth number of soft constraints.

18. The system according to claim 4, wherein the scheduler unit is configured to provide the second number of schedules by adjusting the weightings for at least one further schedule of the second number of schedules and repeating the generating, the displaying, and the calculating for the at least one further schedule.

19. The system according to claim 5, wherein the scheduler unit is configured to improve the first schedule by repeating the selecting, the adjusting, the performing, and the updating for another one of the sixth number of soft constraints.

20. The system according to claim 10, wherein the scheduler unit is configured to at least one of read or store at least one of the schedule database or the constraints database.
